SEQUENCE LISTING (4 sequences)

SEQ ID NO: 1 - DNA, 30 bp, artificial sequence
  Primer
gatagcgggattctagataagggtattagg

SEQ ID NO: 2 - DNA, 25 bp, artificial sequence
  Primer
gcaacaactgcagtgaaatcactgg

SEQ ID NO: 3 - DNA, 957 bp, Escherichia coli
  CDS (1)..(957): rseB sequence
atgaagcaactttggtttgccatgtcattagtgacaggtagcctgttattctctgctaacgcctcggccactcccgcgtccggggcgttattacagcagatgaacctggccagtcagtcactgaattacgagctgtcattcatcagcatcaataaacagggtgttgagtctctgcgttatcgacatgcacgcctcgataaccgtcctcttgcacaattgttgcaaatggatggcccgcgccgggaagtggtacagcgcggcaatgaaatcagctattttgaaccgggacttgaaccgttcacgcttaatggcgattacattgttgattctctgccatcgcttatctataccgatttcaaacgcctttctccttactacgactttatctccgtcgggcgcacgcgtattgctgatcgtctttgcgaagtcattcgcgtggttgcccgagatggtacacgctacagctacatcgtgtggatggacaccgaatcgaaattaccgatgcgggttgatctccttgatcgcgatggtgaaacgctggaacaatttcgcgtgattgcttttaacgtcaatcaggatatcagcagcagtatgcagacgctggcgaaggcaaatttgccgccgttgctttctgttcctgtaggtgaaaaagctaaattcagctggacgccaacctggttgccacagggttttagcgaagtttccagtagtcgacgtccgctaccgacgatggacaacatgcctatcgaatcacgtctctattccgacggattattcagcttctcggtaaacgttaaccgcgctacgccatcgagcaccgatcagatgttgcgcaccggacgcagaaccgtcagtacaagcgtacgtgataacgccgaaatcaccattgtcggtgaactgccgccgcaaacggcgaaacgcattgccgagaatattaagttcggggcagcgcaatga

SEQ ID NO: 4 - protein, 318 aa, Escherichia coli
MKQLWFAMSLVTGSLLFSANASATPASGALLQQMNLASQSLNYELSFISINKQGVESLRYRHARLDNRPLAQLLQMDGPRREVVQRGNEISYFEPGLEPFTLNGDYIVDSLPSLIYTDFKRLSPYYDFISVGRTRIADRLCEVIRVVARDGTRYSYIVWMDTESKLPMRVDLLDRDGETLEQFRVIAFNVNQDISSSMQTLAKANLPPLLSVPVGEKAKFSWTPTWLPQGFSEVSSSRRPLPTMDNMPIESRLYSDGLFSFSVNVNRATPSSTDQMLRTGRRTVSTSVRDNAEITIVGELPPQTAKRIAENIKFGAAQ

(19) United States
(12) Patent Application Publication
(10) Pub. No.: US 2005/0032178 A1
(43) Pub. Date: Feb. 10, 2005

(54) Process for the fermentative preparation of L-amino acids using strains of the enterobacteriaceae family

(75) Inventors: Mechthild Rieping, Bielefeld (DE); Nicole Siebelt, Rietberg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/616,309

(22) Filed: Jul. 10, 2003

Related U.S. Application Data
(60) Provisional application No. 60/395,621, filed on Jul. 15, 2002.

(30) Foreign Application Priority Data
Jul. 10, 2002 (DE) 102 31 115.3

(51) Int. Cl. C12P 13/04; C12P 13/08; C12N 1/21

(52) U.S. Cl. 435/106; 435/115; 435/252.33

(57) ABSTRACT

A process for the fermentive preparation of an L-amino acid, in particular L-threonine, comprising: a) fermentation of a microorganism of the Enterobacteriaceae family which produces the desired L-amino acid and in which the rseB gene or nucleotide sequences which code for it is enhanced, in particular, over-expressed, b) concentration of the desired L-amino acid in the medium or in the cells of the bacteria, and c) isolation or recovery of the desired L-amino acid.

### CROSS-REFERENCE TO RELATED APPLICATIONS

This application claims priority to U.S. Provisional Application No. 60/395,621, filed Jul. 15, 2002 and claims priority to DE (Germany) 102 31 115.3, filed Jul. 10, 2002, the contents of which are hereby incorporated by reference in their entirety.

### REFERENCE TO SEQUENCE LISTING

This application contains a Sequence Listing referring to nucleic acid and amino acid sequences, such as those of the rseB gene. The attached paper copy and computer readable form (on disk) of the sequence listing form part of this disclosure.

### BACKGROUND OF THE INVENTION

1. Field of the Invention

This invention relates to a process for the preparation of L-amino acids, in particular L-threonine, using strains of the Enterobacteriaceae family in which the rseB gene is enhanced.

2. Description of Related Art

L-Amino acids, in particular L-threonine, are used in human medicine and in the pharmaceuticals industry, in the foodstuffs industry and very particularly in animal nutrition.

It is known that L-amino acids are prepared by fermentation of strains of Enterobacteriaceae, in particular Escherichia coli (E. coli )and Serratia marcescens. Because of their great importance, work is constantly being undertaken to improve the preparation processes. Improvements to the process can relate to fermentation measures, such as e.g. stirring and supply of oxygen, or to the composition of the nutrient media, such as e.g. the sugar concentration during the fermentation, or to the working up to the product form, by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), or are auxotrophic for metabolites of regulatory importance and produce L-amino acids, such as e.g. L-threonine, are obtained in this manner.

Methods of the recombinant DNA technique have also been employed for some years for improving strains of the Enterobacteriaceae family which produce L-amino acids, by amplifying individual amino acid biosynthesis genes and investigating the effect on the production. Summarizing information on the cell and molecular biology of Escherichia coli and Salmonella are to be found in Neidhardt (ed): Escherichia coli and Salmonella, Cellular and Molecular Biology, 2^{nd} edition, ASM Press, Washington, D.C., USA (1995).

### BRIEF SUMMARY OF THE INVENTION

The inventors had the object of providing new measures for improved fermentative preparation of L-amino acids, in particular L-threonine.

### BRIEF DESCRIPTION OF THE DRAWING

FIG. 1: Map of the plasmid pTrc99ArseB containing the rseB gene.

### The length data are to be understood as approx. data. The abbreviations and designations used have the following meaning:

Amp: Ampicillin resistance gene
lacI: Gene for the repressor protein of the trc promoter
Ptrc: trc promoter region, IPTG-inducible
rseB: Coding region of the rseB gene
5S: 5S rRNA region
rrnBT: rRNA terminator region
   The abbreviations for the restriction enzymes have the following meaning
EcoRI: Restriction endonuclease from Escherichia coli RY13
EcoRV: Restriction endonuclease from Escherichia coli B946
HindIII: Restriction endonuclease from Haemophilus influenzae
MIul: Restriction endonuclease from Micrococcus luteus IFO 12992
PvuII: Restriction endonuclease from Proteus vulgaris (ATCC 13315)

### DETAILED DESCRIPTION OF THE INVENTION

The invention provides a process for the fermentative preparation of L-amino acids, in particular L-threonine, using microorganisms of the Enterobacteriaceae family which, in particular, already produce L-amino acids and in which the nucleotide sequence which codes for the rseB gene is enhanced.

Where L-amino acids or amino acids are mentioned in the following, this includes one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-threonine is particularly preferred.

The term “enhancement” in this connection describes the increase in the intracellular activity or concentration of one or more enzymes or proteins in a microorganism which are coded by the corresponding DNA, for example by increasing the number of copies of the gene or genes or alleles by at least one (1) copy, using a potent promoter or a gene or allele which codes for a corresponding enzyme or protein with a high activity, and optionally combining these measures.

By enhancement measures, in particular over-expression, the activity or concentration of the corresponding protein is in general increased by at least 1%, 5%, 10%, 25%, 50%, 75%, 100%, 150%, 200%, 300%, 400%, 500%, 1000% or 2000%, based on that of the wild-type protein or the activity or concentration of the protein in the starting microorganism. Variants or mutants of rseB genes may also be selected based on increased functional activity of the gene product which they encode.

The invention provides a process for the preparation
a) of L-amino acids, in particular threonine, or feedstuffs additives comprising these compounds, by culture of microorganisms of the Enterobacteriaceae family in which the rseB gene or nucleotide sequences which code for it are enhanced, in particular over-expressed, in a medium under conditions suitable for the formation of the rseB gene product,
b) the desired L-amino acid can become concentrated in the medium or in the cells, and
c) the product containing the desired L-amino acid(s), is isolated, constituents of the fermentation broth and/or the biomass optionally remaining in the isolated product in an amount of ≧0 to 100%.

The L-amino acids or fermentation products comprising L-amino acids produced by the process of the invention may be incorporated into foods, animal feeds, medical or pharmaceutical products, cosmetic products or other commercial, industrial or consumer products.

The microorganisms, in particular recombinant microorganisms, which the present invention provides can produce L-amino acids from substrates such as glucose, sucrose, lactose, fructose, maltose, molasses, optionally starch, optionally cellulose or from glycerol and ethanol. They are representatives of the Enterobacteriaceae family chosen from the genera Escherichia, Erwinia, Providencia and Serratia. The genera Escherichia and Serratia are preferred. Of the genus Escherichia the species Escherichia coli and of the genus Serratia the species Serratia marcescens are to be mentioned in particular. Recombinant microorganisms are in general generated by transformation, transduction or conjugation with a vector which carries the desired gene.

Suitable strains, which produce L-threonine in particular, of the genus Escherichia, in particular of the species Escherichia coli, are, for example
Escherichia coli H4578 (Applied Micobiology Biotechnology 29: 550-553 (1988))
Escherichia coli KY10935 (Bioscience Biotechnology and Biochemistry 61(11): 1877-1882 (1997))
Escherichia coli VNIIgenetika MG442 (U.S. Pat. No. 4,278,765)
Escherichia coli VNIIgenetika M1 (U.S. Pat. No. 4,321,325)
Escherichia coli VNIIgenetika 472T23 (U.S. Pat. No. 5,631,157)
Escherichia coli BKIIM B-3996 (U.S. Pat. No. 5,175,107)
Escherichia coli kat 13 (WO 98/04715)
Escherichia coli KCCM-10132 (WO 00/09660)
Suitable L-threonine-producing strains of the genus Serratia, in particular of the species Serratia marcescens, are, for example
Serratia marcescens HNr21 (Applied and Environmental Microbiology 38(6): 1045-1051 (1979))
Serratia marcescens TLr156 (Gene 57(2-3): 151-158 (1987))
Serratia marcescens T2000 (Applied Biochemistry and Biotechnology 37(3): 255-265 (1992))

Strains from the Enterobacteriaceae family which produce L-threonine preferably have, inter alia, one or more genetic or phenotypic features chosen from the group consisting of: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin or cyclopentane-carboxylic acid, resistance to rifampicin, resistance to valine analogues, such as, for example, valine hydroxamate, resistance to purine analogues, such as, for example, 6-dimethylaminopurine, a need for L-methionine, optionally a partial and compensatable need for L-isoleucine, a need for meso-diaminopimelic acid, auxotrophy in respect of threonine-containing dipeptides, resistance to L-threonine or threonine raffinate, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally an ability for sucrose utilization, enhancement of the threonine operon, enhancement of homoserine dehydrogenase I-aspartate kinase I, preferably of the feed back resistant form, enhancement of homoserine kinase, enhancement of threonine synthase, enhancement of aspartate kinase, optionally of the feed back resistant form, enhancement of aspartate semialdehyde dehydrogenase, enhancement of phosphoenol pyruvate carboxylase, optionally of the feed back resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

It has been found that microorganisms of the Enterobacteriaceae family produce L-amino acids, in particular L-threonine, in an improved manner after enhancement, in particular over-expression, of the rseB gene.

The nucleotide sequences of the genes of Escherichia coli belong to the prior art (see the following text references) and can also be found in the genome sequence of Escherichia coli published by Blattner et al. (Science 277: 1453-1462 (1997)).

The rseB gene or the protein coded by this gene is described, inter alia, by the following data:

| | |
|---|---|
| Description: | Regulator RseB of sigma-E factor activity |
| Function: | The periplasmic RseB protein regulates sigmaE activity |
| | by interaction with periplasmic C-terminal domains of |
| | the RseA protein. This imparts an anti-sigmaE activity by |
| | inhibition of the transcription of sigmaE-dependent |
| | promoters. |
| Reference: | Missiakas et al.; Molecular Microbiology 24(2), 355-371 |
| | (1997); |
| | De Las Penas et al.; Molecular Microbiology 24(2): |
| | 373-385 (1997); |
| | Collinet et al.; Journal of Biological Chemistry 275(43): |
| | 33898-33904 (2000) |
| Accession No.: | AE000343 |

The nucleic acid sequences can be found in the databanks of the National Center for Biotechnology Information (NCBI) of the National Library of Medicine (Bethesda, Md., USA), the nucleotide sequence databank of the European Molecular Biologies Laboratories (EMBL, Heidelberg, Germany or Cambridge, UK) or the DNA databank of Japan (DDBJ, Mishima, Japan).

The genes described in the text references mentioned can be used according to the invention. For clarity, the nucleotide sequence of the rseB gene or amino acid sequence of the RseB gene product deposited under Accession No. AE000343 is reproduced as SEQ ID No. 3 and 4.

Alleles of the genes which result from the degeneracy of the genetic code or due to at least one (1) “sense mutation” of neutral function can furthermore be used. Alleles which contain sense mutations of neutral function include, inter alia, those which lead to at least one conservative amino acid exchange in the protein coded by them. In the case of aromatic amino acids, conservative exchanges are referred to when phenylalanine, tryptophan and tyrosine are exchanged for one another. In the case of hydrophobic amino acids, conservative exchanges are referred to when leucine, isoleucine and valine are exchanged for one another. In the case of polar amino acids, conservative exchanges are referred to when glutamine and asparagine are exchanged for one another. In the case of basic amino acids, conservative exchanges are referred to when arginine, lysine and histidine are exchanged for one another. In the case of acidic amino acids, conservative exchanges are referred to when aspartic acid and glutamic acid are exchanged for one another. In the case of amino acids containing hydroxyl groups, conservative exchanges are referred to when serine and threonine are exchanged for one another.

Variants of rseB genes may be produced by mutagenesis. Methods of mutagenizing a nucleic acid sequence, such as an rseB gene, are well known and are also described by Current Protocols in Molecular Biology (1987-2002), vols. 1-4, especially vol. 1, chapter 8.

Variants of known rseB genes are also contemplated for use in the present processes. Such variants retain at least one functional activity of a native rseB gene, such as those activities described above for native rseB genes. Preferably a variant should have at least 70%, 80%, 90%, 95% or 99% sequence identity or sequence similarity to a native rseB gene. Such a variant may also be characterized by an ability to hybridize to the nucleic acids encoding an rseB gene, preferably under stringent conditions. Structurally similar nucleic acid sequences, such as those encoding functional RseB polypeptides, may be characterized by their ability to hybridize under stringent conditions to a native rseB nucleic acid sequence. Such hybridization conditions may comprise hybridization at 5×SSC at a temperature of about 50° to 68° C. can be employed for the hybridization reaction. Washing may be performed using 2×SSC and optionally followed by washing using 0.5×SSC. For even higher stringency, the hybridization or washing temperature may be raised to 68° C. or washing may be performed in a salt solution of 0.1×SSC. Other conventional hybridization procedures and conditions may also be used as described by Current Protocols in Molecular Biology, (1987-2003), see e.g., Chapter 2.

Alternatively, variant nucleic acid sequences may be characterized by a particular degree of sequence similarity for instance, at least 60%, 70%, 80%, 90%, 95% or 99% similarity to the nucleic acid sequence of an rseB gene. Such similarity may be determined by an algorithm, such as those described by Current Protocols in Molecular Biology, vol. 4, chapter 19 (1987-2003). Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

In the same way, those nucleotide sequences which code for variants of the proteins mentioned which additionally contain a lengthening or shortening by at least one (1) amino acid on the N or C terminus can also be used. Preferably, this lengthening or shortening is not more than 50, 40, 30, 20, 10, 5, 3 or 2 amino acids or amino acid radicals.

The nucleotide sequence of these genes or alleles or the amino acid sequence of the proteins coded by these genes or alleles is at least 70%, 80%, 85%, 90%, 95%, 96%, 97%, 98% or 99% identical or similar to the sequence of SEQ ID No.3 or 4, the protein having the function of an RseB protein. Sequence identity or similarity may be determined by the use of The use of endogenous genes is preferred. “Endogenous genes” or “endogenous nucleotide sequences” are understood as meaning the genes or alleles or nucleotide sequences present in the population of a species.

To achieve an enhancement, for example, expression of the genes or the catalytic properties of the proteins can be increased. The two measures can optionally be combined.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-threonine production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an over-expression of the genes in question can furthermore be achieved by changing the composition of the media and the culture procedure.

The expert can find instructions in this respect, inter alia, in Chang and Cohen (Journal of Bacteriology 134: 1141-1156 (1978)), in Hartley and Gregori (Gene 13: 347-353 (1981)), in Amann and Brosius (Gene 40: 183-190 (1985)), in de Broer et al. (Proceedings of the National Academy of Sciences of the United States of America 80: 21-25 (1983)), in LaVallie et al. (BIO/TECHNOLOGY 11: 187-193 (1993)), in PCT/US97/13359, in Llosa et al. (Plasmid 26: 222-224 (1991)), in Quandt and Klipp (Gene 80: 161-169 (1989)), in Hamilton et al. (Journal of Bacteriology 171: 4617-4622 (1989)), in Jensen and Hammer (Biotechnology and Bioengineering 58: 191-195 (1998)) and in known textbooks of genetics and molecular biology.

Plasmid vectors which can replicate in Enterobacteriaceae, such as e.g., cloning vectors derived from pACYC184 (Bartolomé et al.; Gene 102: 75-78 (1991)), pTrc99A (Amann et al.; Gene 69: 301-315 (1988)) orpSC101 derivatives (Vocke and Bastia; Proceedings of the National Academy of Sciences USA 80(21): 6557-6561 (1983)) can be used. A strain transformed with a plasmid vector where the plasmid vector carries at least one nucleotide sequence which codes for the rseB gene can be employed in a process according to the invention.

The term transformation is understood in general as meaning the uptake of an isolated nucleic acid by a host (microorganism).

It is also possible to transfer mutations which affect the expression of the particular genes into various strains by sequence exchange (Hamilton et al.; Journal of Bacteriology 171: 4617-4622 (1989)), conjugation or transduction.

More detailed explanations of terms in genetics and molecular biology are found in known textbooks of genetics and molecular biology, such as, for example, the textbook by Birge (Bacterial and Bacteriophage Genetics, 4^{th} ed., Springer Verlag, New York (USA), 2000) or the textbook by Stryer (Biochemistry, 3rd ed., Freeman and Company, New York (USA), 1988) or the handbook by Sambrook et al. (Molecular Cloning, A Laboratory Manual, 2nd ed., Cold Spring Harbor Laboratory Press, Cold Spring Harbor (USA), 1989).

It may furthermore be advantageous for the production of L-amino acids, in particular L-threonine, with strains of the Enterobacteriaceae family to enhance one or more enzymes of the known threonine biosynthesis pathway or enzymes of anaplerotic metabolism or enzymes for the production of reduced nicotinamide adenine dinucleotide phosphate or enzymes of glycolysis or PTS enzymes or enzymes of sulfur metabolism, in addition to the enhancement of the rseB gene. The use of endogenous genes is in general preferred.

Thus, for example, at the same time one or more of the genes chosen from the group consisting of
the thrABC operon which codes for aspartate kinase, homoserine dehydrogenase, homoserine kinase and threonine synthase (U.S. Pat. No. 4,278,765),
the pyc gene of Corynebacterium glutamicum which codes for pyruvate carboxylase (WO 99/18228),
the pps gene which codes for phosphoenol pyruvate synthase (Molecular and General Genetics 231(2): 332-336 (1992)),
the ppc gene which codes for phosphoenol pyruvate carboxylase (Gene 31: 279-283 (1984)),
the pntA and pntB genes which code for transhydrogenase (European Journal of Biochemistry 158: 647-653 (1986)),
the rhtB gene which imparts homoserine resistance (EP-A-0 994 190),
the mqo gene which codes for malate:quinone oxidoreductase (WO 02/06459),
the rhtC gene which imparts threonine resistance (EP-A-1 013 765),
the thrE gene of Corynebacterium glutamicum which codes for the threonine export protein (WO 01/92545),
the gdhA gene which codes for glutamate dehydrogenase (Nucleic Acids Research 11: 5257-5266 (1983); Gene 23: 199-209 (1983)),
the hns gene which codes for the DNA-binding protein HLP-II (WO 03/004671),
the pgm gene which codes for phosphoglucomutase (WO 03/004598),
the fba gene which codes for fructose biphosphate aldolase (WO 03/004664),
the ptsH gene of the ptsHlcrr operon which codes for the phosphohistidine protein hexose phosphotransferase of the phosphotransferase system PTS (WO 03/004674),
the ptsI gene of the ptsHIcrr operon which codes for enzyme I of the phosphotransferase system PTS (WO 03/004674),
the crr gene of the ptsHIcrr operon which codes for the glucose-specific IIA component of the phosphotransferase system PTS (WO 03/004674),
the ptsG gene which codes for the glucose-specific IIBC component (WO 03/004670),
the lrp gene which codes for the regulator of the leucine regulon (WO 03/004665),
the csrA gene which codes for the global regulator Csr (Journal of Bacteriology 175: 4744-4755 (1993)),
the fadR gene which codes for the regulator of the fad regulon (Nucleic Acids Research 16: 7995-8009 (1988)),
the iclR gene which codes for the regulator of the central intermediate metabolism (Journal of Bacteriology 172: 2642-2649 (1990)),
the mopB gene which codes for the 10 Kd chaperone (WO 03/004669) and is also known by the name groES,
the ahpC gene of the ahpCF operon which codes for the small sub-unit of alkyl hydroperoxide reductase (WO 03/004663),
the ahpF gene of the ahpCF operon which codes for the large sub-unit of alkyl hydroperoxide reductase (WO 03/004663),
the cysK gene which codes for cysteine synthase A (WO 03/006666),
the cysB gene which codes for the regulator of the cys regulon (WO 03/006666),
the cysJ gene of the cysJIH operon which codes for the flavoprotein of NADPH sulfite reductase (WO 03/006666),
the cysI gene of the cysJIH operon which codes for the haemoprotein of NADPH sulfite reductase (WO 03/006666),
the cysH gene of the cysJIH operon which codes for adenylyl sulfate reductase (WO 03/006666),
the phoB gene of the phoBR operon which codes for the positive regulator PhoB of the pho regulon (WO 03/008606),
the phoR gene of the phoBR operon which codes for the sensor protein of the pho regulon (WO 03/008606),
the phoE gene which codes for protein E of the outer cell membrane (WO 03/008608),
the pykF gene which codes for fructose-stimulated pyruvate kinase I (WO 03/008609),
the pfkB gene which codes for 6-phosphofructokinase II (WO 03/008610),
the malE gene which codes for the periplasmic binding protein of maltose transport (WO 03/008605),
the sodA gene which codes for superoxide dismutase (WO 03/008613),
the rseA gene of the rseABC operon which codes for a membrane protein with anti-sigmaE activity (WO 03/008612),
the rseC gene of the rseABC operon which codes for a global regulator of the sigmaE factor (WO 03/008612),
the sucA gene of the sucABCD operon which codes for the decarboxylase sub-unit of 2-ketoglutarate dehydrogenase (WO 03/008614),
the sucB gene of the sucABCD operon which codes for the dihydrolipoyltranssuccinase E2 sub-unit of 2-ketoglutarate dehydrogenase (WO 03/008614),
the sucC gene of the sucABCD operon which codes for the β-sub-unit of succinyl-CoA synthetase (WO 03/008615),
the sucD gene of the sucABCD operon which codes for the α-sub-unit of succinyl-CoA synthetase (WO 03/008615),
the adk gene which codes for adenylate kinase (Nucleic Acids Research 13(19): 7139-7151 (1985)),
the hdeA gene which codes for a periplasmic protein with a chaperonin-like function (Journal of Bacteriology 175(23): 7747-7748 (1993)),
the hdeB gene which codes for a periplasmic protein with a chaperonin-like function (Journal of Bacteriology 175(23): 7747-7748 (1993)),
the icd gene which codes for isocitrate dehydrogenase (Journal of Biological Chemistry 262(22): 10422-10425 (1987)),
the mglB gene which codes for the periplasmic, galactose-binding transport protein (Molecular and General Genetics 229(3): 453-459 (1991)),
the lpd gene which codes for dihydrolipoamide dehydrogenase (European Journal of Biochemistry 135(3): 519-527 (1983)),
the aceE gene which codes for the E1 component of the pyruvate dehydrogenase complex (European Journal of Biochemistry 133(1): 155-162 (1983)),
the aceF gene which codes for the E2 component of the pyruvate dehydrogenase complex (European Journal of Biochemistry 133(3): 481-489 (1983)),
the pepB gene which codes for aminopeptidase B (Journal of Fermentation and Bioengineering 82: 392-397 (1996)) and
the aldH gene which codes for aldehyde dehydrogenase (E.C. 1.2.1.3) (Gene 99(1): 15-23 (1991)), can be enhanced, in particular over-expressed.

It may furthermore be advantageous for the production of L-amino acids, in particular L-threonine, in addition to the enhancement of the rseB gene, for one or more of the genes chosen from the group consisting of:
the tdh gene which codes for threonine dehydrogenase (Journal of Bacteriology 169: 4716-4721 (1987)),
the mdh gene which codes for malate dehydrogenase (E.C. 1.1.1.37) (Archives in Microbiology 149: 36-42 (1987)),
the gene product of the open reading frame (orf) yjfA (Accession Number AAC77180 of the National Center for Biotechnology Information (NCBI, Bethesda, Md., USA), WO 02/29080),
the gene product of the open reading frame (orf) ytfP (Accession Number AAC77179 of the National Center for Biotechnology Information (NCBI, Bethesda, Md., USA), (WO 02/29080),
the pckA gene which codes for the enzyme phosphoenol pyruvate carboxykinase (WO 02/29080),
the poxB gene which codes for pyruvate oxidase (WO 02/36797),
the aceA gene which codes for the enzyme isocitrate lyase (WO 02/081722),
the dgsA gene which codes for the DgsA regulator of the phosphotransferase system (WO 02/081721) and is also known under the name of the mlc gene,
the fruR gene which codes for the fructose repressor (WO 02/081698) and is also known under the name of the cra gene,
the rpoS gene which codes for the sigma³⁸ factor (WO 01/05939) and is also known under the name of the katF gene,
the aspA gene which codes for aspartate ammonium lyase (WO 03/008603) and
the aceB gene which codes for malate synthase A (WO 03/008604) to be attenuated, in particular eliminated or for the expression thereof to be reduced.

The term “attenuation” in this connection describes the reduction or elimination of the intracellular activity or concentration of one or more enzymes or proteins in a microorganism which are coded by the corresponding DNA, for example by using a weak promoter or a gene or allele which codes for a corresponding enzyme or protein with a low activity or inactivates the corresponding enzyme (protein) or gene and optionally combining these measures.

By attenuation measures, the activity or concentration of the corresponding protein is in general reduced to 0 to 75%, 0 to 50%, 0 to 25%, 0 to 10% or 0 to 5% the activity or concentration of the wild-type protein or of the activity or concentration of the protein in the starting microorganism.

In addition to enhancement of the rseB gene it may furthermore be advantageous for the production of L-amino acids, in particular L-threonine, to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention can be cultured in the batch process (batch culture), the fed batch (feed process) or the repeated fed batch process (repetitive feed process). A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and optionally cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culturing in a suitable manner.

The fermentation is in general carried out at a pH of 5.5 to 9.0, in particular 6.0 to 8.0. Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of L-amino acids or L-threonine has formed. This target is usually reached within 10 hours to 160 hours.

The analysis of L-amino acids can be carried out by anion exchange chromatography with subsequent ninhydrin derivatization, as described by Spackman et al. (Analytical Chemistry, 30: 1190-1206 (1958)) or it can be carried out by reversed phase HPLC, as described by Lindroth et al. (Analytical Chemistry 51: 1167-1174 (1979)).

The process according to the invention is used for the fermentative preparation of L-amino acids, such as, for example, L-threonine, L-isoleucine, L-valine, L-methionine, L-homoserine and L-lysine, in particular L-threonine.

### EXAMPLES

The present invention is explained in more detail in the following with the aid of embodiment examples.

The minimal (M9) and complete media (LB) for Escherichia coli used are described by J. H. Miller (A short course in bacterial genetics (1992), Cold Spring Harbor Laboratory Press). The isolation of plasmid DNA from Escherichia coli and all techniques of restriction, ligation, Klenow and alkaline phosphatase treatment are carried out by the method of Sambrook et al. (Molecular Cloning—A Laboratory Manual (1989) Cold Spring Harbor Laboratory Press). Unless described otherwise, the transformation of Escherichia coli is carried out by the method of Chung et al. (Proceedings of the National Academy of Sciences of the United States of America 86: 2172-2175 (1989)).

The incubation temperature for the preparation of strains and transformants is 37° C.

### Example 1

Construction of the Expression Plasmid pTrc99ArseB

The rseB gene from E. coli K12 is amplified using the polymerase chain reaction (PCR) and synthetic oligonucleotides. Starting from the nucleotide sequence of the rseB gene in E. coli K12 MG1655 (Accession Number AE000343, Blattner et al. (Science 277: 1453-1474 (1997)), PCR primers are synthesized (MWG Biotech, Ebersberg, Germany).

(SEQ ID No. 1)

| | |
|---|---|
| rseB1: | 5′-GATAGCGGGATTCTAGATAAGGGTATTAGG-3′ |

(SEQ ID No. 2)

| | |
|---|---|
| rseB2: | 5′-GCAACAACTGCAGTGAAATCACTGG-3′ |

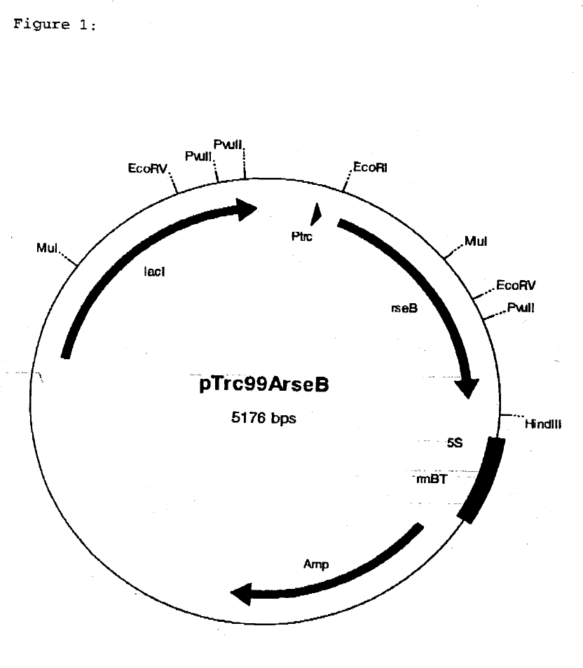

The chromosomal E. coli K12 MG1655 DNA employed for the PCR is isolated according to the manufacturer's instructions with “Qiagen Genomic-tips 100/G” (QIAGEN, Hilden, Germany). A DNA fragment approx. 2100 bp in size can be amplified with the specific primers under standard PCR conditions (Innis et al. (1990) PCR Protocols. A guide to methods and applications, Academic Press) with Pfu-DNA polymerase (Promega Corporation, Madison, USA). The PCR product is cleaved with the restriction enzymes EcoRI and HindIII. After separation in 0.8% agarose gel a fragment approx. 1,000 bp in size is isolated and ligated with the vector pTrc99A (Pharmacia Biotech, Uppsala, Sweden), which has been digested with the enzymes EcoRI and HindIII. The E. coli strain XL1-Blue MRF′ (Stratagene, La Jolla, USA) is transformed with the ligation batch and plasmid-carrying cells are selected on LB agar, to which 50 μg/ml ampicillin are added. Successful cloning can be demonstrated after plasmid DNA isolation by control cleavage with the enzymes EcoRV, MluI and PvuII. The plasmid is called pTrc99ArseB (FIG. 1).

### Example 2

Preparation of L-threonine with the Strain MG442/pTrc99ArseB

The L-threonine-producing E. coli strain MG442 is described in the patent specification U.S. Pat. No. 4,278,765 and deposited as CMIM B-1628 at the Russian National Collection for Industrial Microorganisms (VKPM, Moscow, Russia).

The strain MG442 is transformed with the expression plasmid pTrc99ArseB described in example 1 and with the vector pTrc99A and plasmid-carrying cells are selected on LB agar with 50 μg/ml ampicillin. The strains MG442/pTrc99ArseB and MG442/pTrc99A are formed in this manner. Selected individual colonies are then multiplied further on minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar, 50 mg/l ampicillin. The formation of L-threonine is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose, 50 mg/l ampicillin are inoculated and the batch is incubated for 16 hours at 37° C. and 180 rpm on an ESR incubator from Kühner AG (Birsfelden, Switzerland). 250 μl portions of this preculture are transinoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose, 50 mg/l ampicillin) and the batch is incubated for 48 hours at 37° C. The formation of L-threonine by the starting strain MG442 is investigated in the same manner, but no addition of ampicillin to the medium takes place. After the incubation the optical density (OD) of the culture suspension is determined with an LP2W photometer from Dr. Lange (Düsseldorf, Germany) at a measurement wavelength of 660 nm.

The concentration of L-threonine formed is then determined in the sterile-filtered culture supernatant with an amino acid analyzer from Eppendorf-BioTronik (Hamburg, Germany) by ion exchange chromatography and post-column reaction with ninhydrin detection.

The results of the experiment are shown in Table 1.

TABLE 1

| | | L-Threonine |
|---|---|---|
| Strain | OD | g/l |
| MG442 | 5.6 | 1.4 |
| MG442/pTrc99A | 3.8 | 1.3 |
| MG442/pTrc99ArseB | 5.7 | 2.3 |

Modifications and Other Embodiments

Various modifications and variations of the described genes, gene products, microorganisms and processes, as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the microbiological, molecular biological, chemical, chemical engineering, fermentation arts or related fields are intended to be within the scope of the following claims.

Incorporation by Reference

Each document, patent application or patent publication cited by or referred to in this disclosure is incorporated by reference in its entirety. Any patent document to which this application claims priority is also incorporated by reference in its entirety. Specifically, priority documents U.S. Provisional Application 60/395,621, filed Jul. 15, 2002 and DE (Germany) 102 31 115.3, filed Jul. 10, 2002, are hereby incorporated by reference.

1. A process for the preparation of an L-amino acid comprising:
fermenting a suitable substrate or culture medium with a microorganism of the Enterobacteriaceae family, which comprises an enhanced or overexpressed rseB gene or rseB gene variant, under conditions which are suitable for the expression of the rseB gene or suitable for formation of an rseB gene product, and
isolating or recovering the L-amino acid.

2. The process of claim 1, wherein said microorganism is a recombinant microorganism which is generated by transformation, transduction or conjugation of a microorganism of the Enterobacteriaceae family with a vector, wherein the vector contains an rseB gene or rseB gene variant.

3. The process of claim 1, wherein the number of rseB gene copies in said microorganism is increased by at least one.

4. The process of claim 1, wherein the number of rseB gene copies in said microorganism is increased by at least one and said increase is achieved by integration of the rseB gene into the chromosome of the microorganism.

5. The process of claim 1, wherein the number of rseB gene copies in said microorganism is increased by at least one and said increase is achieved by incorporation of a vector which replicates extrachromosomally into said microorganism.

6. The process of claim 1, wherein over-expression of the rseB gene or rseB gene variant is achieved by:
a) mutation of the promoter and regulation region or the ribosome-binding site upstream of the rseB gene, or
b) incorporation of an expression cassette upstream of the rseB gene or rseB gene variant.

7. The process of claim 1, characterized in that an rseB gene or rseB gene variant which is under the control of a promoter is used.

8. The process of claim 1, wherein said microorganism has at least one additional metabolite or antimetabolite resistance mutation.

9. The process of claim 1, wherein the activity or concentration of the rseB gene product or rseB gene variant product is increased by at least 10%, based on the activity or concentration of the protein in the recipient strain, by the over-expression of the rseB gene.

10. The process of claim 1, wherein said microorganism is selected from the group consisting of the genera Escherichia, Erwinia, Providencia and Serratia.

11. The process of claim 1, wherein said microorganism further comprises one or more gene(s) in which the biosynthesis pathway of the desired L-amino acid is additionally enhanced or over-expressed.

12. The process of claim 1, wherein said microorganism further comprises one or more gene(s) selected from the group consisting of:
the thrABC operon which codes for aspartate kinase, homoserine dehydrogenase, homoserine kinase and threonine synthase,
the pyc gene which codes for pyruvate carboxylase,
the pps gene which codes for phosphoenol pyruvate synthase,
the ppc gene which codes for phosphoenol pyruvate carboxylase,
the pntA and pntB genes which code for transhydrogenase,
the rhtB gene which imparts homoserine resistance,
the mqo gene which codes for malate:quinone oxidoreductase,
the rhtC gene which imparts threonine resistance,
the thrE gene which codes for the threonine export protein,
the gdhA gene which codes for glutamate dehydrogenase,
the hns gene which codes for the DNA-binding protein HLP-II,
the pgm gene which codes for phosphoglucomutase,
the fba gene which codes for fructose biphosphate aldolase,
the ptsH gene which codes for the phosphohistidine protein hexose phosphotransferase,
the ptsI gene which codes for enzyme I of the phosphotransferase system,
the crr gene which codes for the glucose-specific IIA component,
the ptsG gene which codes for the glucose-specific IIBC component,
the lrp gene which codes for the regulator of the leucine regulon,
the csrA gene which codes for the global regulator Csr,
the fadR gene which codes for the regulator of the fad regulon,
the iclR gene which codes for the regulator of central intermediate metabolism,
the mopB gene which codes for the 10 Kd chaperone,
the ahpC gene which codes for the small sub-unit of alkyl hydroperoxide reductase,
the ahpF gene which codes for the large sub-unit of alkyl hydroperoxide reductase,
the cysK gene which codes for cysteine synthase A,
the cysB gene which codes for the regulator of the cys regulon,
the cysJ gene which codes for the flavoprotein of NADPH sulfite reductase,
the cysI gene which codes for the haemoprotein of NADPH sulfite reductase,
the cysH gene which codes for adenylyl sulfate reductase,
the phoB gene which codes for the positive regulator PhoB of the pho regulon,
the phoR gene which codes for the sensor protein of the pho regulon,
the phoE gene which codes for protein E of the outer cell membrane,
the pykF gene which codes for fructose-stimulated pyruvate kinase I,
the pjkB gene which codes for 6-phosphofructokinase II,
the malE gene which codes for the periplasmic binding protein of maltose transport,
the sodA gene which codes for superoxide dismutase,
the rseA gene which codes for a membrane protein with anti-sigmaE activity,
the rseC gene which codes for a global regulator of the sigmaE factor
the sucA gene which codes for the decarboxylase sub-unit of 2-ketoglutarate dehydrogenase,
the sucB gene which codes for the dihydrolipoyltranssuccinase E2 sub-unit of 2-ketoglutarate dehydrogenase,
the sucC gene which codes for the β-sub-unit of succinyl-CoA synthetase,
the sucD gene which codes for the α-sub-unit of succinyl-CoA synthetase,
the adk gene which codes for adenylate kinase,
the hdeA gene which codes for a periplasmic protein with a chaperonin-like function,
the hdeB gene which codes for a periplasmic protein with a chaperonin-like function,
the icd gene which codes for isocitrate dehydrogenase,
the mglB gene which codes for the periplasmic, galactose-binding transport protein,
the lpd gene which codes for dihydrolipoamide dehydrogenase,
the aceE gene which codes for the E1 component of the pyruvate dehydrogenase complex,
the aceF gene which codes for the E2 component of the pyruvate dehydrogenase complex,
the pepB gene which codes for aminopeptidase B and
the aldH gene which codes for aldehyde dehydrogenase, is or are enhanced or over-expressed, are fermented.

13. The process of claim 1, wherein said microorganism has at least one metabolic pathway, which reduces the formation of the desired L-amino acid, eliminated or attenuated.

14. The process of claim 1, wherein said microorganism has attenuated, reduced in expression or eliminated, one or more gene(s) selected from the group consisting of:
the tdh gene which codes for threonine dehydrogenase,
the mdh gene which codes for malate dehydrogenase,
the gene product of the open reading frame (orf) yjfA,
the gene product of the open reading frame (orf) ytfP,
the pckA gene which codes for phosphoenol pyruvate carboxykinase,
the poxB gene which codes for pyruvate oxidase,
the aceA gene which codes for isocitrate lyase,
the dgsA gene which codes for the DgsA regulator of the phosphotransferase system,
the fruR gene which codes for the fructose repressor,
the rpoS gene which codes for the sigma³⁸ factor,
the aspA gene which codes for aspartate ammonium lyase and
the aceB gene which codes for malate synthase A.

15. The process of claim 1, in which
a) the desired L-amino acid is concentrated in the fermentation broth or in the cells of the microorganisms, and
b) the desired product(s) is/are isolated, the biomass and/or further constituents of the fermentation broth optionally remaining in the product in an amount of ≧0 to 100%.

16. The process of claim 1, wherein said L-amino acid is selected from the group consisting of L-asparagine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine.

17. The process of claim 1, wherein said L-amino acid is selected from the group consisting of L-isoleucine, L-valine, L-methionine, L-homoserine and L-lysine.

18. The process according to claim 1, wherein said L-amino acid is L-threonine.

19. A recombinant microorganism of the Enterobacteriaceae family in which the rseB gene, rseB gene variant, or nucleotide sequences which code for the rseB gene product are present in over-expressed form.

20. A microorganism according to claim 19, which produces L-threonine.
A process for the fermentative preparation of L-amino acids, in particular L-threonine, in which the following steps are carried out:
a) fermentation of the microorganisms of the Enterobacteriaceae family which produce the desired L-amino acid and in which the rseB gene or nucleotide sequences which code for it are enhanced, in particular over-expressed,
b) concentration of the desired L-amino acid in the medium or in the cells of the bacteria, and
c) Isolation or recovery of the desired L-amino acid.
